(19) United States
(12) Patent Application Publication
(10) Pub. No.: US 2023/0255607 A1
(43) Pub. Date: Aug. 17, 2023

(54) TISSUE SAMPLE DEVICE AND METHODS

(71) Applicant: BOSTON SCIENTIFIC SCIMED, INC., Maple Grove, MN (US)

(72) Inventors: Richard Crawford, Galway (IE); Enda Connaughton, Galway (IE); Peter Brady, Galway (IE); Hugh Edward Mulcahy, Dublin (IE)

(73) Assignee: BOSTON SCIENTIFIC SCIMED, INC., Maple Grove, MN (US)

(21) Appl. No.: 18/109,664

(22) Filed: Feb. 14, 2023

Related U.S. Application Data
(60) Provisional application No. 63/309,818, filed on Feb. 14, 2022.

(51) Int. Cl. A61B 10/02

(52) U.S. Cl. CPC A61B 10/02; A61B 2010/0216

(57) ABSTRACT

A tissue collection system for collection a sample from the body. The system includes an outer tubular member having a proximal end region to a distal end region and defining a lumen extending from the proximal end region to the distal end region, an inner tubular member slidably disposed within the lumen of the outer tubular member, the inner tubular member defining a lumen extending from a proximal end region to a distal end region of the inner tubular member, and a tissue collection device disposed adjacent to the distal end region of the inner tubular member.

### CROSS-REFERENCE TO RELATED APPLICATIONS

This application claims the benefit of priority under 35 U.S.C. § 119 of U.S. Provisional Application No. 63/309,818, filed Feb. 14, 2022, the entire disclosure of which is hereby incorporated by reference.

### TECHNOLOGY FIELD

The present application relates generally to medical devices for tissue sample collection, and more particularly, to medical devices for tissue sample collection resulting in greater cellular or other tissue yield.

### BACKGROUND

Certain medical tests require sampling of cells from target areas of a subject's body. For instance, a screening test for detecting potentially pre-cancerous and cancerous tissues in a subject's body may include taking samples of tissue or cells from a target area of the subject's body. A tissue collection device may be used to collect cells or other tissues from the target area. Tissue collection from some parts of the anatomy may be difficult. There is an ongoing need to provide alternative medical devices as well as alternative methods for manufacturing and using medical devices.

### SUMMARY

This disclosure provides design, material, manufacturing methods, and use alternatives for medical devices.

In a first example, a tissue collection system may comprise an outer tubular member having a proximal end region and a distal end region and defining a lumen extending from the proximal end region to the distal end region, an inner tubular member slidably disposed within the lumen of the outer tubular member, the inner tubular member defining a lumen extending from a proximal end region to a distal end region of the inner tubular member, and a tissue collection device disposed adjacent to the distal end region of the inner tubular member. The inner tubular member may be movable between a retracted delivery position and an extended sample collection position.

Alternatively or additionally to any of the examples above, in another example, the tissue collection device may be disposed about an outer surface of the inner tubular member.

Alternatively or additionally to any of the examples above, in another example, the tissue collection device may be removably coupled to the distal end region of the inner tubular member.

Alternatively or additionally to any of the examples above, in another example, the distal end region of the inner tubular member may comprise a coupling mechanism.

Alternatively or additionally to any of the examples above, in another example, the coupling mechanism of the inner tubular member may comprise a plurality of threads.

Alternatively or additionally to any of the examples above, in another example, the tissue collection device may comprise an elongate shaft extending from a distal end region to a proximal end region and defining a lumen extending from the distal end region to the proximal end region.

Alternatively or additionally to any of the examples above, in another example, the proximal end region of the tissue collection device may comprise a coupling mechanism.

Alternatively or additionally to any of the examples above, in another example, the coupling mechanism of the tissue collection device may comprise a plurality of threads.

Alternatively or additionally to any of the examples above, in another example, the coupling mechanism of the tissue collection device may be configured to releasably couple to the coupling mechanism of the inner tubular member.

Alternatively or additionally to any of the examples above, in another example, the tissue collection device may further comprise a radially extending seal mechanism positioned adjacent to the proximal end region of the tissue collection device.

Alternatively or additionally to any of the examples above, in another example, the tissue collection system may further comprise an atraumatic distal tip member disposed distal to the tissue collection device.

Alternatively or additionally to any of the examples above, in another example, at least a portion of the atraumatic distal tip member may be configured to contact a distal end of the outer tubular member when the inner tubular member is in a retracted delivery configuration.

Alternatively or additionally to any of the examples above, in another example, the tissue collection device may include a brush mechanism.

Alternatively or additionally to any of the examples above, in another example, the brush mechanism may comprise a plurality of radially extending bristles.

Alternatively or additionally to any of the examples above, in another example, the plurality of radially extending bristles may be grouped into a plurality of brush clusters.

Alternatively or additionally to any of the examples above, in another example, the tissue collection system may further comprise a guidewire, the guidewire may be configured to be slidably disposed within the lumen of the inner tubular member in a co-axial arrangement with the inner tubular member.

In another example, a tissue collection system may comprise an outer tubular member having a proximal end region and a distal end region and defining a lumen extending from the proximal end region to the distal end region, an inner tubular member slidably disposed within the lumen of the outer tubular member, the inner tubular member defining a lumen extending from a proximal end region to a distal end region of the inner tubular member, and a tissue collection device disposed about and extending radially from the distal end region of the inner tubular member. The inner tubular member may be movable between a retracted delivery position and an extended sample collection position.

Alternatively or additionally to any of the examples above, in another example, the tissue collection device may include a brush mechanism.

Alternatively or additionally to any of the examples above, in another example, the tissue collection system may further comprise an atraumatic distal tip member disposed distal to the tissue collection device.

Alternatively or additionally to any of the examples above, in another example, at least a portion of the atraumatic distal tip member may be configured to contact a distal end of the outer tubular member when the inner tubular member is in a retracted delivery configuration.

In another example, a tissue collection system may comprise an outer tubular member having a proximal end region and a distal end region and defining a lumen extending from the proximal end region to the distal end region, an inner tubular member slidably disposed within the lumen of the outer tubular member, the inner tubular member defining a lumen extending from a proximal end region to a distal end region of the inner tubular member, and a tissue collection device releasably coupled to the distal end region of the inner tubular member. The inner tubular member may be movable between a retracted delivery position and an extended sample collection position.

Alternatively or additionally to any of the examples above, in another example, the tissue collection device may comprise an elongate shaft extending from a distal end region to a proximal end region and defining a lumen extending from the distal end region to the proximal end region, a brush mechanism extending radially from an outer surface of the elongate shaft, and an atraumatic distal tip member disposed adjacent to the distal end region.

Alternatively or additionally to any of the examples above, in another example, the proximal end region of the tissue collection device may comprise a coupling mechanism.

Alternatively or additionally to any of the examples above, in another example, the coupling mechanism of the tissue collection device may be configured to releasably couple to a mating coupling mechanism of the inner tubular member.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 illustrates a partial cross-sectional side view of an illustrative tissue collection device system for delivering a tissue collection device to a target region in a retracted or delivery configuration;

FIG. 2 illustrates a partial cross-sectional side view of the illustrative tissue collection device system of FIG. 1 with the tissue collection device in a deployed or collection configuration;

FIG. 3 illustrates a cross-sectional view of a distal end region of another illustrative tissue collection device delivery system; and

FIG. 4 is an illustrative flow chart of a method for collecting a tissue sample using the systems of FIGS. 1-3.

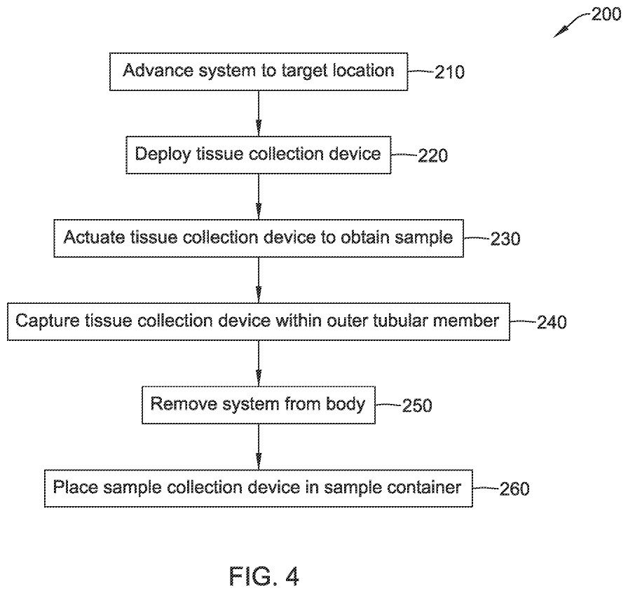

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION OF EXEMPLARY EMBODIMENTS

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features, and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

Endoscopic retrograde cholangiopancreatography (ERCP) is a procedure that utilizes both endoscopic and fluoroscopic techniques to diagnose and treat issues arising in the common bile duct (CBD) and pancreatic ducts (PD). One of the main issues being treated today is strictures in the CBD from such as, but not limited to, primary sclerosing cholangitis (PSC), cancer of the bile duct, damage and scarring due to a gallstone in the bile duct, etc. ERCP has now matured into predominantly an interventional procedure. When a clinician needs to take a sample of the stricture, one of the most common ways to do this is by using a cytology brush.

At present, there is no satisfactory cytology brush mechanism with which to take cellular samples from common bile duct strictures. Clinicians may use stricture brushing, with or without intraductal aspiration of bile. However, achieving a brush position over the stricture remains a problem in many cases. The tip of the mechanism will frequently not pass through the ampulla and/or the stricture. Current tissue collection devices or brushes may include devices having snub-nosed outer catheter tips. The guidewire may be advanced through a separate channel of the catheter that is adjacent to the brush rather than inside the brush. As a result, when being inserted, the catheter tip may pass around curved surfaces and through strictures with difficulty, being inclined to impact on the outer surface of the bend. What may be desirable is a tissue collection device and system which allows the tip of the delivery catheter and the tissue collection device to pass the ampulla, bends, and stricture with ease. While the present disclosure is described with respect to the common bile duct and pancreatic ducts, the devices and methods are not limited to such use. For example, the devices and methods described herein may be used in any portion of the anatomy, as desired. Further, the devices and methods described herein may be used either endoscopic or non-endoscopic anatomies. Some illustrative anatomies may include, but are not limited to, the mouth, the esophagus, the stomach, the duodenum, other portions of the gastrointestinal tract, the pathways leading to the lungs, other portions of the respiratory system, the urinary tract, the cervix, other reproductive anatomy, etc.

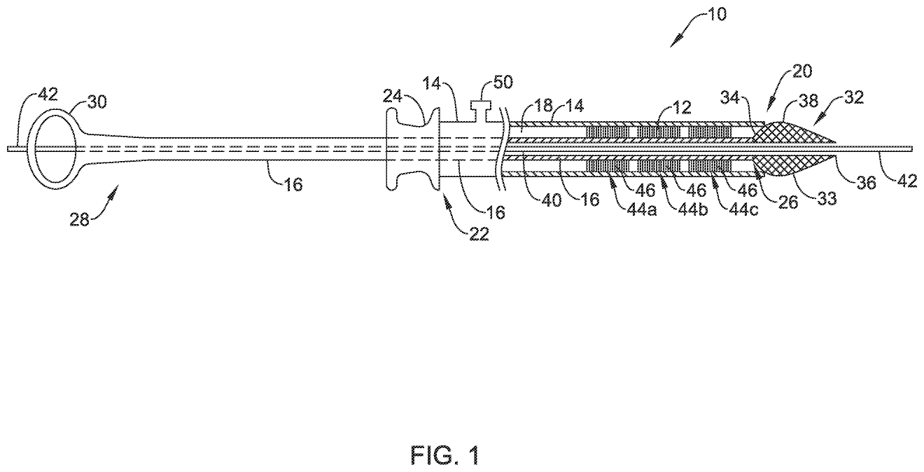

FIG. 1 is a partial cross-sectional side view of an illustrative tissue collection device system 10 for delivering a tissue collection device 12 to a target region, such as, but not limited to, the common bile duct or pancreatic duct, in a retracted or delivery configuration. The tissue collection system 10 may include an outer or exterior elongate shaft or tubular member 14 and an inner elongate shaft or tubular member 16. The inner tubular member 16 may be slidably disposed within a lumen 18 of the outer tubular member 14. The outer tubular member 14 may extend proximally from a distal end region 20 to a proximal end region 22 configured to remain outside of a patient's body. A first hub or handle 24 may be coupled to the proximal end region 22 of the outer tubular member 14. In some cases, a port 50, such as an injection port, may be provided in the outer tubular member 14. Other structures to facilitate connection to other medical devices (e.g., syringe, stopcocks, Y-adapter, etc.) and to provide access to lumen 18 may be provided. The inner tubular member 16 may extend proximally from a distal end region 26 to a proximal end region 28 configured to remain outside of a patient's body. A second hub or handle 30 may be coupled to the proximal end region 28 of the inner tubular member 16.

The outer tubular member 14 may include a lumen 18 extending from the distal end region 20 to the proximal end region 22. The lumen 18 may also extend through the first handle 24. The lumen 18 of the outer tubular member 14 and the first handle 24 may be configured to slidably receive the inner tubular member 16. The inner tubular member 16 may include a lumen 40 extending from the distal end region 26 to the proximal end region 28. The lumen 40 of the inner tubular member 16 may also extend through the second handle 30. The lumen 40 of the inner tubular member 16 may be configured to receive a guidewire 42, as desired.

The distal end region 26 of the inner tubular member 16 may include an atraumatic distal tip member 32. The atraumatic distal tip member 32 may be sized and shaped to facilitate advancement of the inner tubular member 16 and the tissue collection device 12 through bends and strictures. The atraumatic distal tip member 32 may include a body 33 having a proximal end 34, a distal end 36, and an intermediate region 38. The body 33 may be solid with the lumen 40 of the inner tubular member 16 extending therethrough. The cross-sectional dimension of the atraumatic distal tip member 32 in a direction generally orthogonal to a longitudinal axis of the inner tubular member 16 may vary along a length of the atraumatic distal tip member 32. For example, the cross-sectional dimension may increase in the distal direction from the proximal end 34 towards the intermediate region 38 and decrease from the intermediate region 38 to the distal end 36. Said differently, a maximum cross-sectional dimension of the atraumatic distal tip member 32 may be at or near the intermediate region 38. It is contemplated that such a configuration may allow the proximal end 34 of the atraumatic distal tip member 32 to be disposed within the lumen 18 of the outer tubular member 14 during delivery and removal of the device. The intermediate region 38 may have a cross-sectional dimension that is similar to or greater than an inner diameter of the outer tubular member 14 such that the intermediate region 38 may abut, contact, or be adjacent to the distal end region 20 of the outer tubular member 14 to seal the lumen 18 thereof. As will be described in more detail herein, this may help reduce sample loss during retraction of the system 10.

The tissue collection device 12 may be disposed around a portion of the inner tubular member 16 at or adjacent to the distal end region 26 thereof. The tissue collection device 12 may extend about an entirety of the circumference of the inner tubular member 16. In other embodiments, the tissue collection device 12 may be radially spaced about a circumference of the inner tubular member 16 in a uniform pattern or eccentric manner, as desired. In some embodiments, the tissue collection device 12 may include a brush mechanism including a plurality of bristles 46. While the tissue collection device 12 is described as a brush mechanism, it is contemplated that other tissue collection devices may be used, as desired. The bristles 46 may extend radially outward from an outer surface of the inner tubular member 16. The bristles 46 may be arranged in a first brush cluster 44a, a second brush cluster 44b, and a third brush cluster 44c (collectively, 44). While the tissue collection device 12 is illustrated as including three longitudinally spaced brush clusters 44, the tissue collection device 12 may include fewer than three (e.g., one or two) or more than three (e.g., four, five, six, or more) brush clusters 44. The bristles 46 may be coupled, adhered, or otherwise affixed to an outer surface of the inner tubular member 16. In some cases, the bristles 46 may be coupled directly to the outer surface of the inner tubular member 16. Alternatively, the bristles 46 may be affixed to one or more collars or rings and then the collars or rings coupled to the outer surface of the inner tubular member 16. It is contemplated that a collar or ring may be a solid component or may be a plurality of wires twisted into a cylindrical tube in which the bristles 46 may be clamped between the twisted wires.

The bristles 46 may be employed to brush against a tissue surface in the target area to capture cells. The bristles 46 may be arranged/mounted around the distal portion of the inner tubular member 16. In some embodiments, each of the bristles 46 may have a cross-section that is substantially circular. However, the bristles 46 may have any other suitable cross-sectional shape, including rectangular, triangular, square, polygonal, elliptical, or oblong.

The bristles 46 may be made of one or more filaments. For example, a plurality of the bristles 46 may be made of a continuous length of a filament. Alternatively, a plurality of the bristles 46 may be made of separate lengths of a filament. The filament may be a monofilament. The monofilament may be formed by extrusion. Alternatively, the filament may be a multicomponent filament. A multicomponent filament may include a core about which one or more layers of material are concentrically arranged. If multiple layers are present, they may differ in composition and/or thickness. The outermost one of the layers may include micropatterning, as will be described in more detail below. The multicomponent filament may be formed by coextrusion. It is contemplated that the filament may be made of nylon, polymer, and/or any suitable material or combination of materials.

It is contemplated that the bristles 46 may be arranged in any configuration desired. In some cases, the bristles 46 may be arranged helically around the distal portion of the inner tubular member 16, and may extend radially outwards from the inner tubular member. In some embodiments, the bristles 46 may radiate at an angle relative to the longitudinal axis of the inner tubular member 16. It is further contemplated that a density of the bristles 46 may vary along a length of the tissue collection device 12 or between clusters 44a, 44b, 44c. In some cases, a length of the bristles 46 may varied. When the bristles 46 are brushed against tissue in the target area, cells from the tissue may be transferred to the bristles 46, and may be captured between the bristles 46.

When the tissue collection device 12 is disposed within the outer tubular member 14, the tissue collection device 12 may be restrained in a compressed reduced diameter or delivery configuration by the outer tubular member 14 surrounding the tissue collection device 12. In the compressed configuration, the tissue collection device 12 may have a smaller diameter than the expanded deployed configuration. The distal end region 20 of the outer tubular member 14 may be positioned such that the outer tubular member 14 surrounds and covers the length of the tissue collection device 12 during delivery. The outer tubular member 14 may have sufficient hoop strength to retain the tissue collection device 12 in its reduced diameter state.

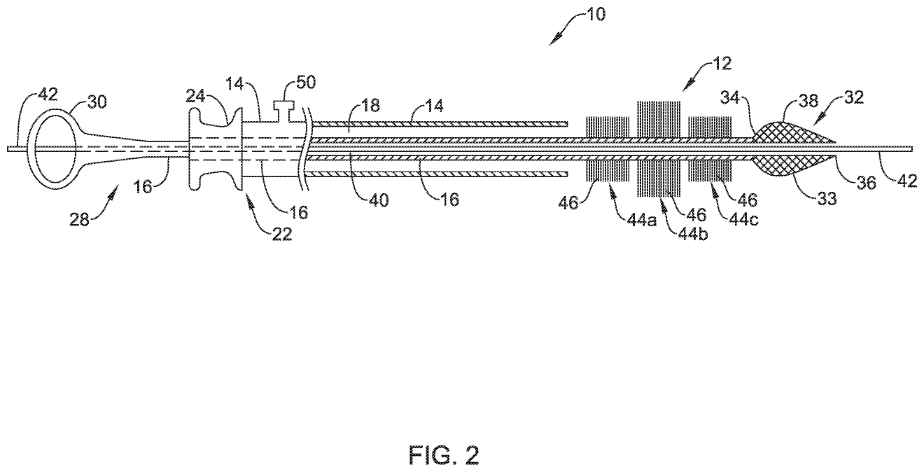

FIG. 2 is a partial cross-sectional side view of the illustrative tissue collection device system 10 of FIG. 1 with the tissue collection device 12 in a deployed or collection configuration. The tissue collection system 10 may be advanced through the body towards the target location, as desired. The tissue collection system 10 may be advanced with or without the use of a guidewire 42. Once the tissue collection device 12 is positioned adjacent to the target region, the restraining forces maintaining the tissue collection device 12 in the radially compressed configuration may be removed to deploy the tissue collection device 12.

The tissue collection device 12 may be deployed by actuating the second handle 30, for example, by distally pushing the second handle 30, while maintaining the first handle 24 in a fixed position. Thus, the inner tubular member 16 may be distally advanced relative to the outer tubular member 14. In other words, the inner tubular member 16 may be distally advanced while the outer tubular member 14 is held stationary. The reverse configuration is also contemplated. For example, the outer tubular member 14 may be proximally retracted while the inner tubular member 16 is held stationary. As shown in FIG. 2, as the inner tubular member 16 is distally advanced, the biasing force is removed from the exterior of the tissue collection device 12 and the tissue collection device 12 assumes its radially expanded, unbiased, deployed configuration.

As can be seen in FIG. 2, the first brush cluster 44a and the third brush cluster 44c have a first height and the second brush cluster 44b has a second height greater than the first height. It is contemplated that the other height combinations of the bristles 46 or brush clusters 44 may be used as desired. For example, the height of the bristles 46 may increase in a tapered, sloped, or stepped manner in the distal direction. Alternatively, the height of the bristles 46 may decrease in a tapered, sloped, or stepped manner in the distal direction. In yet another example, the height of the bristles 46 may increase and decrease in an undulating pattern along a length of the tissue collection device 12. These are just some examples. The height of the bristles 46 may be arranged in any configuration desired.

Once the tissue collection device 12 is deployed from the outer tubular member 14, the second handle 30 may be actuated to repeatedly distally advance, proximally retract, and/or rotate the tissue collection device 12 along the target collection site. This may cause the bristles 46 to brush against the tissue surface to capture cells among the bristles 46. Once the clinician has captured cells from the target site, the inner tubular member 16 may be proximally retracted until the tissue collection device 12 is disposed within the lumen 18 of the outer tubular member 14 and the atraumatic distal tip member 32 is in contact with the distal end of the outer tubular member 14 which may seal the distal opening of the outer tubular member 14. Sealing of the distal opening of the outer tubular member 14 may prevent the collected sample from dissipating during removal of the tissue collection system 10 from the body.

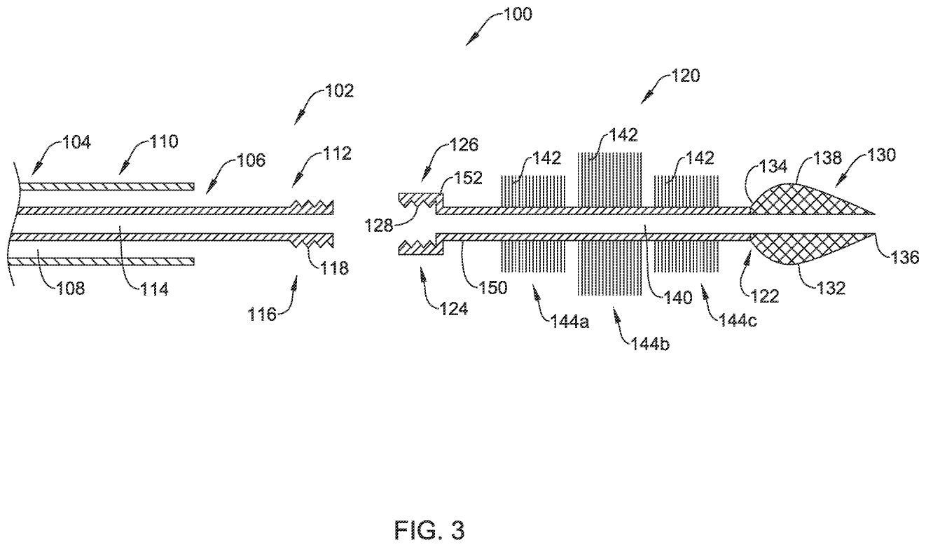

FIG. 3 is a cross-sectional view of a distal end region 102 of another illustrative tissue collection device system 100 having a removable tissue collection device 120. In FIG. 3, the tissue collection device 120 is illustrated as uncoupled or detached from the inner tubular member 106. However, it should be understood that during delivery of the system 100 and sample collection, the tissue collection device 120 is coupled to the inner tubular member 106.

The tissue collection system 100 may include an outer or exterior elongate shaft or tubular member 104 and an inner elongate shaft or tubular member 106. The inner tubular member 106 may be slidably disposed within a lumen 108 of the outer tubular member 104. The outer tubular member 104 may extend proximally from a distal end region 110 to a proximal end region (not explicitly shown) configured to remain outside of a patient's body. While not explicitly shown, a first hub or handle similar in form and function to the first handle 24 of FIGS. 1 and 2 may be coupled to the proximal end region of the outer tubular member 104. The inner tubular member 106 may extend proximally from a distal end region 112 to a proximal end region (not explicitly shown) configured to remain outside of a patient's body. A second hub or handle similar in form and function to the second handle 30 of FIGS. 1 and 2 may be coupled to the proximal end region of the inner tubular member 106.

The outer tubular member 104 may include a lumen 108 extending from the distal end region 110 to the proximal end region. The lumen 108 may also extend through the first handle. The lumen 108 of the outer tubular member 104 and the first handle may be configured to slidably receive the inner tubular member 106. The inner tubular member 106 may include a lumen 114 extending from the distal end region 112 to the proximal end region. The lumen 114 of the inner tubular member 106 may also extend through the second handle. The lumen 114 of the inner tubular member 106 may be configured to receive a guidewire (not explicitly shown), as desired.

The distal end region 112 of the inner tubular member 106 may include a coupling mechanism 116 configured to releasably secure the inner tubular member 106 to a tissue collection device 120. In the illustrated embodiment, the coupling mechanism 116 may include a plurality of external threads 118 formed on an outer surface of the inner tubular member 106. The external threads 118 may be configured to mate with corresponding threads 128 on the tissue collection device 120. It is contemplated that other releasably coupling mechanisms may be used, as desired, such as, but not limited to, a friction fit, a snap fit (such as, but not limited to a mating detent and groove), a bayonet style coupling mechanism, a quick release coupling mechanism, a spring-loaded hook, etc.

The tissue collection device 120 may include an elongate shaft 150 extending from a distal end region 122 to a proximal end region 124. The tissue collection device 120 may include a lumen 140 extending from the distal end region 122 to the proximal end region 124. When the tissue collection device 120 is coupled to the inner tubular member 106, the lumen 140 may be fluid communication with the lumen 114 of the inner tubular member. The lumen 140 of the tissue collection device 120 may be configured to receive a guidewire, as desired.

The proximal end region 124 of the tissue collection device 120 may include a coupling mechanism 126 configured to be releasably secured with the coupling mechanism 116 of the inner tubular member 106. The coupling mechanism 126 may include a plurality of internal threads 128 configured to mate with corresponding threads 118 on the inner tubular member 106. It is contemplated that the threading configuration may be reversed such that the internal threads are located on the inner tubular member 106 and the external threads are located on the tissue collection device 120. It is contemplated that other releasably coupling mechanisms may be used, as desired, such as, but not limited to, a friction fit, a snap fit (such as, but not limited to a mating detent and groove), a bayonet style coupling mechanism, a quick release coupling mechanism, a spring-loaded hook, etc. The coupling mechanism 126 of the tissue collection device 120 may include a radially extending gasket or seal mechanism 152 proximal to the plurality of bristles 142. The seal mechanism 152 may have an outer diameter that is sized to allow the tissue collection 120 to slide within the lumen of the outer tubular member 104 while substantially blocking fluid flow through the lumen 108. For example, the outer diameter of the seal mechanism 152 may be approximately the same as an inner diameter of the outer tubular member 104 or less than an inner diameter of the outer tubular member 104. It is contemplated that if the seal mechanism 152 and the inner surface of the outer tubular member 104 are suitably lubricious, the outer diameter of the seal mechanism 152 may be greater than the inner diameter of the outer tubular member 104. While the seal mechanism 152 has been described as extending from the coupling mechanism 126, it is contemplated that the seal mechanism 152 may extend from the elongate shaft 150 if so desired.

The distal end region 122 of the tissue collection device 120 may include an atraumatic distal tip member 130. The atraumatic distal tip member 130 may include a body 132 having a proximal end 134, a distal end 136, and an intermediate region 138. The body 132 may be solid with the lumen 140 of the tissue collection device 120 extending therethrough. The cross-sectional dimension of the atraumatic distal tip member 130 in a direction generally orthogonal to a longitudinal axis of the tissue collection device 120 may vary along a length of the atraumatic distal tip member 130. For example, the cross-sectional dimension may increase in the distal direction from the proximal end 134 towards the intermediate region 138 and decrease from the intermediate region 138 to the distal end 136. Said differently, a maximum cross-sectional dimension of the atraumatic distal tip member 130 may be at or near the intermediate region 138. It is contemplated that such a configuration may allow the proximal end 134 of the atraumatic distal tip member 130 to be disposed within the lumen 108 of the outer tubular member 104 during delivery and removal of the device. The intermediate region 138 may have a cross-sectional dimension that is similar to or greater than an inner diameter of the outer tubular member 104 such that the intermediate region 138 may abut, contact, or be adjacent to the distal end region 110 of the outer tubular member 104 to seal the lumen 108 thereof. As will be described in more detail herein, this may help reduce sample loss during retraction of the system 100.

In some embodiments, the tissue collection device 120 may include a brush mechanism including a plurality of bristles 142. While the tissue collection device 120 is described as a brush mechanism, it is contemplated that other tissue collection devices may be used, as desired. The bristles 142 may extend radially outward from an outer surface of the elongate shaft 150. The bristles 142 may be arranged in a first brush cluster 144a, a second brush cluster 144b, and a third brush cluster 144c (collectively, 144). While the tissue collection device 120 is illustrated as including three longitudinally spaced brush clusters 144, the tissue collection device 120 may include fewer than three (e.g., one or two) or more than three (e.g., four, five, six, or more) brush clusters 144. The bristles 142 may be coupled, adhered, or otherwise affixed to an outer surface of the inner tubular member 106. In some cases, the bristles 142 may be coupled directly to the outer surface of the inner tubular member 106. Alternatively, the bristles 142 may be affixed to one or more collars or rings and then the collars or rings coupled to the outer surface of the inner tubular member 106. It is contemplated that a collar or ring may be a solid component or may be a plurality of wires twisted into a cylindrical tube in which the bristles 142 may be clamped between the twisted wires.

The first brush cluster 144a and the third brush cluster 144c have a first height and the second brush cluster 144b has a second height greater than the first height. It is contemplated that the other height combinations of the bristles 142 or brush clusters 144 may be used as desired. For example, the height of the bristles 142 may increase in a tapered, sloped, or stepped manner in the distal direction. Alternatively, the height of the bristles 142 may decrease in a tapered, sloped, or stepped manner in the distal direction. In yet another example, the height of the bristles 142 may increase and decrease in an undulating pattern along a length of the tissue collection device 120. These are just some examples. The height of the bristles 142 may be arranged in any configuration desired.

The bristles 142 may be employed to brush against a tissue surface in the target area to capture cells. The bristles 142 may be arranged/mounted around an intermediate region of the elongate shaft 150. In some embodiments, each of the bristles 142 may have a cross-section that is substantially circular. However, the bristles 142 may have any other suitable cross-sectional shape, including rectangular, triangular, square, polygonal, elliptical, or oblong.

The bristles 142 may be made of one or more filaments. For example, a plurality of the bristles 142 may be made of a continuous length of a filament. Alternatively, a plurality of the bristles 142 may be made of separate lengths of a filament. The filament may be a monofilament. The monofilament may be formed by extrusion. Alternatively, the filament may be a multicomponent filament. A multicomponent filament may include a core about which one or more layers of material are concentrically arranged. If multiple layers are present, they may differ in composition and/or thickness. The outermost one of the layers may include micropatterning, as will be described in more detail below. The multicomponent filament may be formed by coextrusion. It is contemplated that the filament may be made of nylon, polymer, and/or any suitable material or combination of materials.

It is contemplated that the bristles 142 may be arranged in any configuration desired. In some cases, the bristles 142 may be arranged helically around the distal portion of the inner tubular member 106, and may extend radially outwards from the inner tubular member. In some embodiments, the bristles 142 may radiate at an angle relative to the longitudinal axis of the inner tubular member 106. It is further contemplated that a density of the bristles 142 may vary along a length of the tissue collection device 120 or between clusters 144a, 144b, 144c. In some cases, a length of the bristles 142 may varied. When the bristles 142 are brushed against tissue in the target area, cells from the tissue may be transferred to the bristles 142, and may be captured between the bristles 142.

In some embodiments, the inner tubular member 106 and the tissue collection device 120 may be formed from a same material. In other embodiments, the inner tubular member 106 and the tissue collection device 120 may be formed from different materials. For example, the tissue collection device 120 may be formed from a material which facilitates the attachment, coupling, or formation of the bristles 142.

When the tissue collection device 120 is disposed within the outer tubular member 104, the tissue collection device 120 may be restrained in a compressed reduced diameter or delivery configuration by the outer tubular member 104 surrounding the tissue collection device 120. In the compressed configuration (not explicitly shown), the tissue collection device 120 may have a smaller diameter than the expanded deployed configuration. The distal end region 110 of the outer tubular member 104 may be positioned such that the outer tubular member 104 surrounds and covers the length of the tissue collection device 120 during delivery. The outer tubular member 104 may have sufficient hoop strength to retain the tissue collection device 120 in its reduced diameter state.

The tissue collection system 100 may be advanced through the body towards the target location, as desired. It should be understood that during delivery of the system 100 and sample collection, the tissue collection device 120 is coupled to the inner tubular member 106. The tissue collection system 100 may be advanced with or without the use of a guidewire. Once the tissue collection device 120 is positioned adjacent to the target region, the restraining forces maintaining the tissue collection device 120 in the radially compressed configuration may be removed to deploy the tissue collection device 120.

The tissue collection device 120 may be deployed by actuating the second handle, for example, by distally pushing the second handle, while maintaining the first handle in a fixed position. Thus, the inner tubular member 106 may be distally advanced relative to the outer tubular member 104. In other words, the inner tubular member 106 may be distally advanced while the outer tubular member 104 is held stationary. The reverse configuration is also contemplated. For example, the outer tubular member 104 may be proximally retracted while the inner tubular member 106 is held stationary. As the inner tubular member 106 is distally advanced, the biasing force is removed from the exterior of the tissue collection device 120 and the tissue collection device 120 assumes its radially expanded, unbiased, deployed configuration.

Once the tissue collection device 120 is deployed from the outer tubular member 104, the second handle may be actuated to repeatedly distally advance, proximally retract, and/or rotate the tissue collection device 120 along the target collection site. This may cause the bristles 142 to brush against the tissue surface to capture cells among the bristles 142. Once the clinician has captured cells from the target site, the inner tubular member 106 may be proximally retracted until the tissue collection device 120 is disposed within the lumen 108 of the outer tubular member 104 and the atraumatic distal tip member 130 is in contact with the distal end of the outer tubular member 104. The seal mechanism 152 may engage an inner wall of the outer tubular member 104 and the distal tip member 130 may seal the distal opening of the outer tubular member 104. Sealing of the proximal end region 124 of the tissue collection device 120 and the distal opening of the outer tubular member 104 may prevent the collected sample from dissipating (e.g., into the space between the inner and outer tubular members 106, 104) during removal of the tissue collection system 100 from the endoscope and/or body.

FIG. 5 is an illustrative flow chart of a method 200 for collecting a tissue sample using the systems 10, 100 of FIGS. 1-3. To begin, the tissue collection system 10, 100 may be advanced to the target location in the body, as shown at block 210. In some embodiments, the tissue collection system 10, 100 may be advanced through an endoscope, a distal end of which may be positioned near the target location. For example, to obtain a sample from the common bile duct, an endoscope may be advanced through the esophagus, through the stomach, and into the duodenum. The tissue collection system 10, 100 may be positioned within the endoscope as it is being positioned or may be subsequently advanced therethrough. The guidewire 42 of the tissue collection system 10, 100 may then be distally advanced to cannulate the common bile duct. The inner tubular member 16, 106 may then be distally advanced over the guidewire to deploy the tissue collection device 12, 120, as shown at block 220. As described above, the inner tubular member 16, 106 may be distally advanced while the outer tubular member 14, 104 is stationary such that the tissue collection device 12, 120 exits the outer tubular member 14, 104. It is contemplated that as the guidewire 42 is centrally located within the tissue collection system 10, 100, the inner tubular member 16, 106 is not biased to one side as with a tissue collection system which advances a brush through a separate channel from the guidewire. The co-axial arrangement of the guidewire 42 and the inner tubular member 16, 106 may allow the atraumatic distal tip member 32, 130 and tissue collection device to pass the ampulla, bends, and stricture with ease.

Once the tissue collection device 12, 120 has been positioned at the target location, the inner tubular member 16, 106 may be actuated (e.g., using the second handle 30) back and forth (e.g., proximally and distally) to drag the bristles 46, 142 along the collection site to gather cells, as shown at block 230. In some cases, the inner tubular member 16, 106 may be rotated in addition to or alternatively to proximal and distal movement. Once the sample has been collected, the inner tubular member 16, 106 may be proximally retracted to draw the tissue collection device 12, 120 back into the lumen 108 of the outer tubular member 14, 104, as shown at block 240. The inner tubular member 16, 106 may be retracted until the atraumatic distal tip member 32, 130 is in contact with the distal end of the outer tubular member 14, 104. As described above, the atraumatic distal tip member 32, 130 may block or close off the distal opening of the outer tubular member 14, 104 to help reduce sample loss during removal of the tissue collection system 10, 100 from the body. When so provided, the seal mechanism 152 may create a proximal seal to further reduce sample loss. Once the atraumatic distal tip member 32, 130 is positioned against the distal end of the outer tubular member 14, 104, the tissue collection system 10, 100 may be removed from the body, as shown at block 250. The inner tubular member 16 may then be cut (e.g., using wire cutters or other cutting device) at a location proximal to the tissue collection device 12 such that the tissue collection device 12 may be placed into a sample container, as shown at block 260. Alternatively, the tissue collection device 120 may be uncoupled from the inner tubular member 106 by actuating the coupling mechanisms 116, 126. It is contemplated that if another sample is desired, another tissue collection device 120 may be coupled to the coupling mechanism 116 of the previously used inner tubular member 106. This may help reduce cost and waste by allowing multiple cytology passes to be made using a single tissue collection system but using two or more tissue collection devices 120. It may also simplify the process of cutting the tissue collection device using a wire cutter for placement into the sample container, since the tissue collection device 120 would detach from the inner tubular member 106 by actuating the coupling mechanisms 116, 126 (e.g., unscrewing in the illustrated example. Alternatively, with the tissue collection system 10 of FIGS. 1 and 2, an entirely new inner tubular member 16 assembly and tissue collection device 12 must be used for each desired sample.

The materials that can be used for the various components of the medical device system 10, 100 (and/or other systems disclosed herein) and the various elements thereof disclosed herein may include those commonly associated with medical devices. For simplicity purposes, the following discussion refers to the tissue collection system 10, 100 and/or the tissue collection device 12, 120. However, this is not intended to limit the devices and methods described herein, as the discussion may be applied to other elements, members, components, or devices disclosed herein, such as, but not limited to, the inner and outer tubular members 16, 106, 14, 104, handles 24, 30, guidewire 42, distal tip members 32, 130, etc. and/or elements or components thereof.

In some embodiments, tissue collection system 10, 100, the tissue collection device 12, 120, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of tissue collection system 10, 100, the tissue collection device 12, 120, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device system 10 to achieve the same result.

In some embodiments, a degree of Magnetic Resonance Imaging (MRI) compatibility is imparted into the medical device system 10. For example, tissue collection system 10, 100, the tissue collection device 12, 120, and/or components or portions thereof, may be made of a material that does not substantially distort the image and create substantial artifacts (e.g., gaps in the image). Certain ferromagnetic materials, for example, may not be suitable because they may create artifacts in an MRI image. The tissue collection system 10, 100, the tissue collection device 12, 120, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, an exterior surface of the medical device system 10 (including, for example, an exterior surface of the delivery system) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the outer sheath, or in embodiments without an outer sheath over portions of the delivery system, or other portions of the medical device system 10. Hydrophobic coatings such as fluoropolymers provide a dry lubricity which improves device handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A tissue collection system, the system comprising:
an outer tubular member having a proximal end region and a distal end region and defining a lumen extending from the proximal end region to the distal end region;
an inner tubular member slidably disposed within the lumen of the outer tubular member, the inner tubular member defining a lumen extending from a proximal end region to a distal end region of the inner tubular member; and
a tissue collection device disposed adjacent to the distal end region of the inner tubular member;
wherein the inner tubular member is movable between a retracted delivery position and an extended sample collection position.

2. The tissue collection system of claim 1, wherein the tissue collection device is disposed about an outer surface of the inner tubular member.

3. The tissue collection system of claim 1, wherein the tissue collection device is removably coupled to the distal end region of the inner tubular member.

4. The tissue collection system of claim 3, wherein the distal end region of the inner tubular member comprises a coupling mechanism.

5. The tissue collection system of claim 4, wherein the tissue collection device comprises an elongate shaft extending from a distal end region to a proximal end region and defining a lumen extending from the distal end region to the proximal end region.

6. The tissue collection system of claim 5, wherein the proximal end region of the tissue collection device comprises a coupling mechanism.

7. The tissue collection system of claim 6, wherein the coupling mechanism of the tissue collection device is configured to releasably couple to the coupling mechanism of the inner tubular member.

8. The tissue collection system of claim 5, the tissue collection device further comprising a radially extending seal mechanism positioned adjacent to the proximal end region of the tissue collection device.

9. The tissue collection system of claim 1, further comprising an atraumatic distal tip member disposed distal to the tissue collection device.

10. The tissue collection system of claim 9, wherein at least a portion of the atraumatic distal tip member is configured to contact a distal end of the outer tubular member when the inner tubular member is in a retracted delivery configuration.

11. The tissue collection system of claim 1, wherein the tissue collection device includes a brush mechanism.

12. The tissue collection system of claim 1, further comprising a guidewire, the guidewire configured to be slidably disposed within the lumen of the inner tubular member in a co-axial arrangement with the inner tubular member.

13. A tissue collection system, the system comprising:
an outer tubular member having a proximal end region and a distal end region and defining a lumen extending from the proximal end region to the distal end region;
an inner tubular member slidably disposed within the lumen of the outer tubular member, the inner tubular member defining a lumen extending from a proximal end region to a distal end region of the inner tubular member; and
a tissue collection device disposed about and extending radially from the distal end region of the inner tubular member;
wherein the inner tubular member is movable between a retracted delivery position and an extended sample collection position.

14. The tissue collection system of claim 13, wherein the tissue collection device includes a brush mechanism.

15. The tissue collection system of claim 13, further comprising an atraumatic distal tip member disposed distal to the tissue collection device.

16. The tissue collection system of claim 15, wherein at least a portion of the atraumatic distal tip member is configured to contact a distal end of the outer tubular member when the inner tubular member is in a retracted delivery configuration.

17. A tissue collection system, the system comprising:
an outer tubular member having a proximal end region and a distal end region and defining a lumen extending from the proximal end region to the distal end region;
an inner tubular member slidably disposed within the lumen of the outer tubular member, the inner tubular member defining a lumen extending from a proximal end region to a distal end region of the inner tubular member; and
a tissue collection device releasably coupled to the distal end region of the inner tubular member;
wherein the inner tubular member is movable between a retracted delivery position and an extended sample collection position.

18. The tissue collection system of claim 17, wherein the tissue collection device comprises:
an elongate shaft extending from a distal end region to a proximal end region and defining a lumen extending from the distal end region to the proximal end region;
a brush mechanism extending radially from an outer surface of the elongate shaft; and
an atraumatic distal tip member disposed adjacent to the distal end region.

19. The tissue collection system of claim 17, wherein the proximal end region of the tissue collection device comprises a coupling mechanism.

20. The tissue collection system of claim 19, wherein the coupling mechanism of the tissue collection device is configured to releasably couple to a mating coupling mechanism of the inner tubular member.
